(19) United States
(12) Patent Application Publication
(10) Pub. No.: US 2024/0009080 A1
(43) Pub. Date: Jan. 11, 2024

(54) PHARMACEUTICAL CONTAINER

(71) Applicant: SCHOTT Pharma AG & Co. KGaA, Mainz (DE)

(72) Inventors: Matthias Bicker, Mainz (DE); Christoph Brüning, Kiedrich (DE); Anne Spende, Wiesbaden (DE); Flor Toledo Rodríguez, Frankfurt am Main (DE); Michaela Klause, Mainz (DE); Uwe Rothhaar, Birkenheide (DE); Tanja Woywod, Mainz (DE); Eveline Rudigier-Voigt, Mainz (DE); Jovana Djordjevic-Reiss, Mainz (DE); Sylvia Biedenbender, Bingen (DE); Harmut Bauch, Mommenheim (DE)

(73) Assignee: SCHOTT Pharma AG & Co. KGaA, Mainz (DE)

(21) Appl. No.: 18/471,763

(22) Filed: Sep. 21, 2023

Related U.S. Application Data
(63) Continuation of application No. PCT/EP2022/058033, filed on Mar. 25, 2022.

(30) Foreign Application Priority Data
Mar. 25, 2021 (EP) 21164784.7
Mar. 25, 2021 (EP) 21164787.0
Mar. 25, 2021 (EP) 21164788.8
Mar. 25, 2021 (EP) 21164881.1
Mar. 25, 2021 (EP) 21164896.9
Mar. 25, 2022 (WO) PCT/EP2022/058033

(51) Int. Cl. A61J 1/14

(52) U.S. Cl. CPC A61J 1/1468

(57) ABSTRACT

A pharmaceutical container includes an inner surface and an outer surface. At least part of the inner surface is coated with a coating to form a coated inner surface. On the coated inner surface the container, based on negative mode Time-of-Flight-Secondary Ion Mass Spectrometry (ToF-SIMS) data, has a relative lipid nanoparticle (LNP)-incubated Multivariate Curve Resolution (MCR) score ratio of lipid factor1 of less than 0.67.

### CROSS REFERENCE TO RELATED APPLICATIONS

This is a continuation of International Patent Application No. PCT/EP2022/058033 filed on Mar. 25, 2022, which is incorporated in its entirety herein by reference. International Patent Application No. PCT/EP2022/05803 claims priority to European Patent Application No. 21164784.7 filed on Mar. 25, 2021, which is incorporated in its entirety herein by reference. International Patent Application No. PCT/EP2022/05803 also claims priority to European Patent Application No. 21164787.0 filed on Mar. 25, 2021, which is incorporated in its entirety herein by reference. International Patent Application No. PCT/EP2022/05803 also claims priority to European Patent Application No. 21164788.8 filed on Mar. 25, 2021, which is incorporated in its entirety herein by reference. International Patent Application No. PCT/EP2022/05803 also claims priority to European Patent Application No. 21164881.1 filed on Mar. 25, 2021, which is incorporated in its entirety herein by reference. International Patent Application No. PCT/EP2022/05803 also claims priority to European Patent Application No. 21164896.9 filed on Mar. 25, 2021, which is incorporated in its entirety herein by reference.

### BACKGROUND OF THE INVENTION

### 1. Field of the Invention

The invention relates to a pharmaceutical container. The container is particularly suited for the storage and transportation of pharmaceutical compositions with sensitive ingredients, such as e.g. mRNA-LNP based drug products.

### 2. Description of the Related Art

Lipid-based carrier systems, such as lipid nanoparticles (LNPs), are a modern drug delivery vehicle that is used for pharmaceutically active, sensitive ingredients, such as e.g. mRNA.

LNPs used for mRNA vaccines against SarS-CoV-2 are based chemically different types of lipids, e.g. phospholipids, cholesterol, PEG-modified lipids and cationic lipids. Cationic lipids bind mRNA due to their opposite molecular charges. mRNA molecules are chemically sensitive and require high demands on their storage conditions, for example, in some instances, temperatures well below −20° C. to preserve the drug.

Buschmann et al. (Vaccines 9, 2021, 65) described an overview of mRNA delivery systems with a focus on lipid nanoparticles used in the then current SARS-CoV-2 vaccine clinical trials.

Accordingly, high demands are placed on the containers for the storage and transport of mRNA vaccines. RNA-based active agents are highly potent drugs, requiring only very small dosages. As of today, these medicines are available in multi-dose containers. It is important that each dose drawn from the container contains the same amount of active agent, and that the active agent is present in the container in its original form even after long terms of storage.

Accordingly, there remains a need to provide a container for pharmaceutically active, sensitive ingredients which diminishes and/or avoids the adhesion and possible inactivation of lipid-based carrier systems.

### SUMMARY OF THE INVENTION

In some embodiments, this disclosure relates to a pharmaceutical container comprising an inner surface and an outer surface, wherein at least part of the inner surface is coated with a coating, wherein on the coated inner surface the container, based on negative mode ToF-SIMS data, has a relative lipid nanoparticle (LNP)-incubated MCR score ratio of lipid factor1 of less than 0.67, less than 0.5, less than 0.3 or less than 0.13.

In some embodiments, this disclosure relates to a pharmaceutical container comprising an inner surface and an outer surface, wherein at least part of the inner surface is coated with a coating, wherein on the coated inner surface the container, based on negative mode ToF-SIMS data, has an absolute LNP-incubated MCR score of lipid factor1 of less than 7×10¹³, less than 5×10¹³, or less than 2×10¹³.

In some embodiments, this disclosure relates to a pharmaceutical container comprising an inner surface and an outer surface, wherein at least part of the inner surface is coated with a coating, wherein on the coated inner surface the container, based on negative mode ToF-SIMS data, has an absolute LNP-incubated MCR score of silicon-organic factor1 of at least 1×10¹².

In some embodiments, this disclosure relates to a pharmaceutical container comprising an inner surface and an outer surface, wherein at least part of the inner surface is coated with a coating, wherein on the coated inner surface the container, based on negative mode ToF-SIMS data, has a relative LNP-incubated MCR score ratio of silicon-organic factor1 of at least 2, or at least 5.

In some embodiments, this disclosure relates to a pharmaceutical container comprising an inner surface and an outer surface, wherein at least part of the inner surface is coated with a coating, wherein on the coated inner surface the container has an LNP-incubated haze value of less than 50%, or less than 30%, measured according to the ASTM D 1003-13 standard using illuminant D65 and 2° observer, wherein the LNP-incubated haze value is obtained after freezing to −80° C., and incubating for 4 weeks at −80° C.

The container provided according to this disclosure is suitable for pharmaceutical compositions and overcomes the problems associated with containers known in the state of the art. The container allows storage and transportation of pharmaceutical compositions, such as compositions comprising lipid-based carrier systems, such as lipid nanoparticles, and specifically mRNA, or siRNA or saRNA containing formulations, including vaccines. The container overcomes the problem(s) of multi-dose uniformity and preservation of the pharmaceutical composition in its original form, even after long terms of storage.

Whereas a vast variety of coated containers are already known, there has remained a challenge to provide pharmaceutical containers that are suitable for the storage and transport of compositions comprising lipid-based carrier systems, and lipid nanoparticles in particular. The subject-matter provided according to this disclosure meets this desire by providing a container with a coating which has improved adhesion repellent properties. In this context, “improved adhesion” means decreased adhesion. Even after long terms of storage, adhesion of the constituents of the lipid-based carrier systems is very low. Thus, dose-uniformity is excellent and the pharmaceutical composition remains intact and unaltered.

The improved adhesion properties of the container are embodied and expressed by virtue of the factors and their scores obtained using MCR as described herein-below.

In some embodiments, the invention relates to a filled pharmaceutical container comprising the pharmaceutical container provided according to this disclosure, and a pharmaceutical composition comprising lipid-based carrier systems, in particular lipid nanoparticles.

In some embodiments, this disclosure relates to a pharmaceutical container comprising an inner surface and an outer surface, wherein at least part of the inner surface is coated with a coating, wherein on the coated inner surface the container, based on negative mode ToF-SIMS data, particularly without LNP-incubation, has an absolute MCR score of silicon-organic factor1 of at least at least 3×10¹², and/or a relative MCR score ratio of silicon-organic factor1 of at least 2; or an absolute MCR score of silicon-inorganic factor1 of up to 3×10¹³, and/or a relative MCR score ratio of silicon-inorganic factor1 is up to 5.

The filled pharmaceutical container may advantageously allow for excellent dose uniformity and inertness towards lipid-based carrier systems because it shows less adhesion of ingredients of pharmaceutical composition to the inner surface of the container. Advantageously, when the pharmaceutical composition comprises lipid-based carrier systems, in particular lipid nanoparticles, there may be less adhesion of the lipids and/or lipid nanoparticles to the inner surface of the container.

In some embodiments, the invention relates to the use of the pharmaceutical container for the storage and/or transport of pharmaceutical compositions comprising lipid-based carrier systems, in particular lipid nanoparticles.

### BRIEF DESCRIPTION OF THE DRAWINGS

The above-mentioned and other features and advantages of this invention, and the manner of attaining them, will become more apparent and the invention will be better understood by reference to the following description of embodiments of the invention taken in conjunction with the accompanying drawings, wherein:

FIG. 1 shows photographs obtained from an uncoated glass vial and a coated glass vial, wherein the glass vial was manufactured with glass tubing (Fiolax® clear, Schott AG, Germany), subjected to a reference solution and a solution containing LNPs;

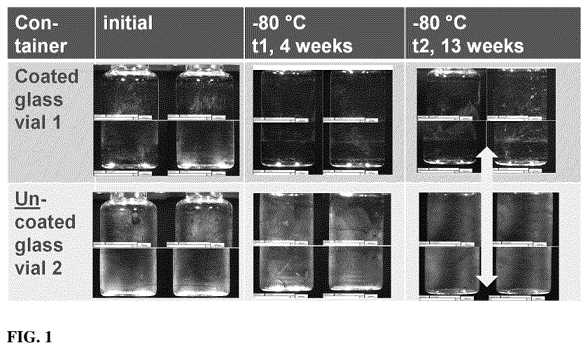

FIG. 2A shows the loading for the characteristic lipid factor1 obtained from the data matrix of negative-ToF-SIMS spectra for adsorbed lipid-containing compounds on the inner surface of glass vials;

FIG. 2B shows the score values obtained for coated and uncoated glass vials from the MCR analysis based on the lipid factor1 with loading shown in FIG. 2A, each experiment was carried out in duplicate, both on the bottom wall and on the bottom-near wall of the inner surface of the glass vials;

FIG. 3A shows the loading for the characteristic lipid factor1 obtained from the data matrix of negative-ToF-SIMS spectra for adsorbed lipid-containing compounds on the inner surface of coated and uncoated polymer syringes made of COC-polymer;

FIG. 3B shows the score values obtained for coated and uncoated polymer syringes from the MCR analysis based on the lipid factor with loading in FIG. 3A, each experiment was carried out in duplicate, both on the bottom wall and on the bottom-near wall of the inner surface;

FIG. 4A shows the loading for the characteristic silicon-organic factor1 obtained from the data matrix of negative-ToF-SIMS spectra for silicon-organic compounds on the inner surface of coated and uncoated glass vials, glass syringes and polymer syringes;

FIG. 4B shows the score values obtained for coated and uncoated containers from the MCR analysis of the data matrix shown in FIG. 4A, each experiment was carried out in duplicate, both on the bottom wall and on the bottom-near wall of the inner surface;

FIG. 5A shows the list of ion species which are selected from the raw ToF-SIMS data including their intensities, obtained from a glass container;

FIG. 5B shows the list of ion species which are selected from the raw ToF-SIMS data including their intensities, obtained from a polymer container;

FIG. 6A shows the loading for the characteristic silicon-inorganic factor1 obtained from the data matrix of negative-ToF-SIMS spectra for silicon-inorganic compounds on the inner surface of coated and uncoated glass vials;

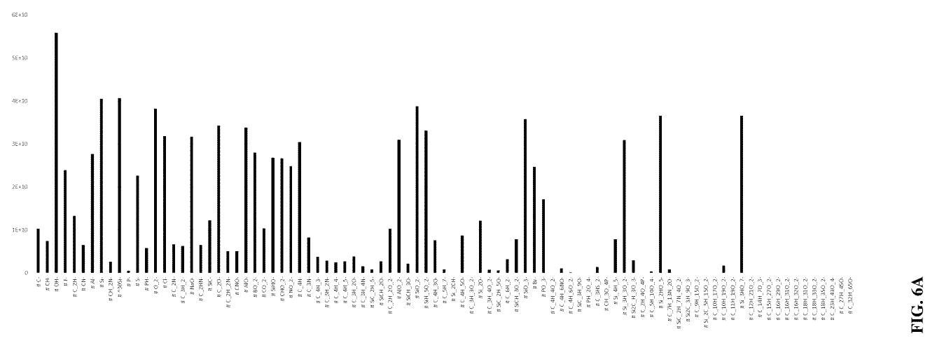

FIG. 6B shows the score values obtained for coated and uncoated containers from the MCR analysis of the data matrix shown in FIG. 6A, each experiment was carried out in duplicate, both on the bottom wall and on the bottom-near wall of the inner surface;

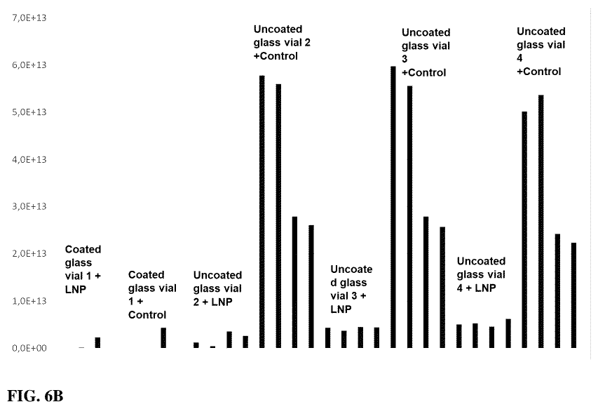

FIG. 7A shows the loading for the characteristic organic factor1 obtained from the data matrix of negative-ToF-SIMS spectra for organic compounds on the inner surface of coated and uncoated glass vials; and

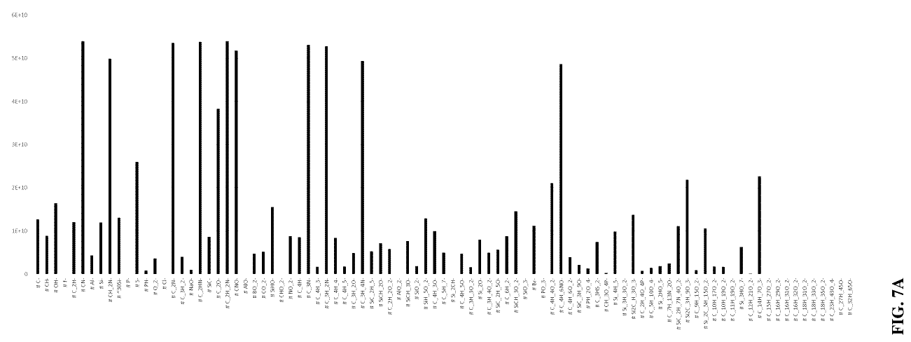

FIG. 7B shows the score values obtained for coated and uncoated glass vials from the MCR analysis of the data matrix shown in FIG. 7A, each experiment was carried out in duplicate, both on the bottom wall and on the bottom-near wall of the inner surface.

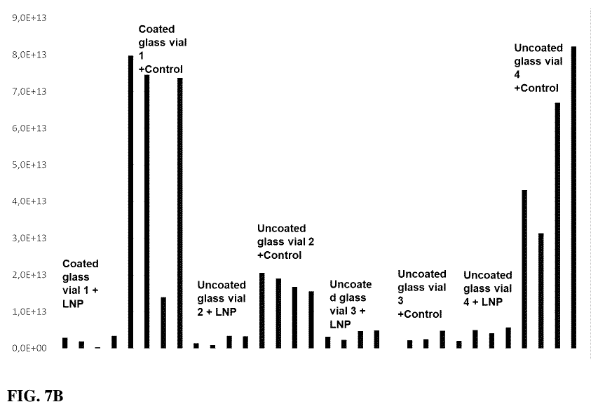

Corresponding reference characters indicate corresponding parts throughout the several views. The exemplifications set out herein illustrate embodiments of the invention and such exemplifications are not to be construed as limiting the scope of the invention in any manner.

### DETAILED DESCRIPTION OF THE INVENTION

The pharmaceutical container provided according to the invention may be a syringe, a cartridge, an ampoule or a vial. The container may be a glass container, such as a borosilicate glass container, an aluminosilicate glass container or a boroaluminosilicate glass container. Alternatively, the pharmaceutical container may be manufactured from a suitable polymer, such as cycloolefinic copolymer (COC) or cycloolefinic polymer (COP). The inner surface of the pharmaceutical container is coated with a coating which provides for desirable surface properties with respect to the adhesion of lipid-based carrier systems, such as lipid nanoparticles (LNPs). For the purposes of establishing the factors and scores of this disclosure, the coated container is subjected to a negative-mode ToF-SIMS data acquisition and a subsequent MCR (Multivariate Curve Resolution) analysis, as further outlined in detail below. Any reference to “LNP incubated” in this disclosure means that the container or coating was incubated with LNPs before measurement. If a score is denoted as a “relative” score ratio, the respective values are to be understood as being the relative ratio of the score value of the coated container divided by the score value of an uncoated reference container based on the same MCR factor. E.g., for measuring a relative LNP-incubated MCR score ratio between a coated container and an uncoated container, wherein the uncoated container is a reference container, both containers are incubated with the same specific LNP composition as applied for the coated container. Both the coated container and the reference container are analysed via ToF-SIMS and MCR to obtain absolute MCR scores, e.g. of lipid factor1. The relative LNP-incubated MCR score ratio, e.g. of lipid factor1, is obtained by dividing the resulting MCR score of the coated container by the MCR score of the reference container. For example, the relative LNP-incubated MCR score ratio of lipid factor1 may be less than 0.5. As mentioned before, the reference container may be an uncoated container. The reference container may be of the same dimensions and materials and bulk composition as the coated container (except for the coating of course).

Advantageously, the coated container may be less prone to adhesion of lipids, which is expressed in a low relative LNP-incubated MCR score ratio of lipid factor1 when comparing the coated container to the reference container.

In some embodiments, this disclosure provides for a pharmaceutical container comprising an inner surface and an outer surface, wherein at least part of the inner surface is coated with a coating, wherein on the coated inner surface the container, based on negative-mode ToF-SIMS data, has a relative LNP-incubated MCR score ratio of lipid factor1 of less than 0.5, wherein the coated container is compared to a reference container, wherein the relative LNP-incubated MCR score ratio of lipid factor1 is obtained by dividing the absolute MCR score of lipid factor1 of the coated container by the absolute MCR score of lipid factor1 of the uncoated container.

In some embodiments, LNP-incubation of a glass container, either being an uncoated glass container or a coated glass container, comprises cleaning the container with UltraPure water (purity 1 analogue DIN ISO 3696 with ≤0.1 μS/cm at 25° C.), drying under laminar-flow conditions, incubating the container with a reference LNP-composition by filling the container with the reference LNP-composition, freezing to −80° C., incubating for 12 hours at −80° C., and then thawing to 5° C. within 12 hours, and then emptying the containing followed by a cleaning step of the inner container surface by rinsing 10 times with ultrapure water and subsequent drying under laminar flow.

In some embodiments, LNP-incubation of a polymer container, either being an uncoated polymer container or a coated polymer container, comprises incubating the container with a reference LNP-composition by filling the container with the reference LNP-composition, freezing to −80° C., incubating for 12 hours at −80° C., and then thawing to 5° C. within 12 hours and then emptying the containing followed by a cleaning step of the inner container surface by rinsing 10 times with ultrapure water and subsequent drying under laminar flow.

In some embodiments, the reference LNP-composition is the Comirnaty® vaccine drug product (license number EU/1/20/1528).

In some embodiments, the reference LNP-composition contains the following lipids in the indicated amounts: 7.2 mg/mL (4-hydroxybutyl)azanediyl)bis(hexane-6,1-diyl)bis(2-hexyldecanoate), 0.83 mg/mL 2[(polyethylene glycol)-2000]-N,N-ditetradecylacetamide, 1.5 mg/mL 1,2-distearoyl-sn-glycero-3-phosphocholine, and 3.3 mg/mL cholesterol, in phosphate-buffered saline (PBS) (pH 7.4) with a sucrose content of 10 wt. % and the following concentrations.

| Compound | Concentration | Concentration |
|---|---|---|
| (salt or sugar) | (mmol/L) | (g/L) |
| NaCl | 137 | 8.0 |
| KCl | 2.7 | 0.2 |
| Na₂HPO₄ | 10 | 1.42 |
| KH₂PO₄ | 1.8 | 0.24 |
| Sucrose | | 100 |

In some embodiments, the container has an absolute LNP-incubated MCR score of lipid factor1 of less than 7×10¹³, less than 5×10¹³, or less than 2×10¹³, or even less than 0.5×10¹³.

In some embodiments, the container has a relative LNP-incubated MCR score ratio of lipid factor1 of less than 0.67, less than 0.5, less than 0.3 or less than 0.13.

In some embodiments, the container has an absolute LNP-incubated MCR score of silicon-organic factor1 of at least 1×10¹², at least 2×10¹², or at least 3×10¹². Optionally, the absolute LNP-incubated MCR score of silicon-organic factor1 may reach up to 9×10¹³, up to 7×10¹³, or up to 6×10¹³.

In some embodiments, the container has a relative LNP-incubated MCR score ratio of silicon-organic factor1 of at least 2, at least 3, or at least 5. Optionally, the relative LNP-incubated MCR score ratio of silicon-organic factor1 may be up to 20, up to 15, or up to 10. It was found that an MCR score of silicon-organic factor1 in this range decreases lipid- and in particular LNP-adhesion to a remarkable extent.

In some embodiments, the pharmaceutical container has an absolute LNP-incubated MCR score of silicon-inorganic factor1 of up to 1×10¹³, up to 5×10¹², or up to 3×10¹². Optionally, this score may be at least 0.5×10¹².

In some embodiments, the relative LNP-incubated MCR score ratio of silicon-inorganic factor1 is up to 5, up to 3, or up to 1.5. Optionally, this score ratio is at least 0.1, or at least 0.2.

In some embodiments, the container has an absolute LNP-incubated MCR score of organic factor1 of at least 1×10¹², at least 2×10¹², or at least 3×10¹² and/or a relative LNP-incubated MCR score ratio of organic factor1 of at least 0.2, at least 0.5, or at least 1.0. Optionally, the relative LNP-incubated MCR score ratio of organic factor1 is up to 10, up to 5, up to 2.0.

Optionally, the container has an absolute LNP-incubated MCR score of organic factor1 of up to 9×10¹², up to 8×10¹², or up to 6×10¹².

Alternatively or in addition, the container may have corresponding, non-LNP-incubated score values. These values are obtained without LNP-incubation (“non-incubated”).

An absolute non-incubated MCR score of silicon-organic factor1 may be at least 3×10¹², at least 5×10¹², or at least 7×10¹². Optionally, the absolute non-incubated MCR score of silicon-organic factor1 may reach up to 9×10¹³, up to 7×10¹³, or up to 6×10¹³.

A relative non-incubated MCR score ratio of silicon-organic factor1 may be at least 3×10¹², at least 5×10¹², or at least 7×10¹². Optionally, the absolute LNP-incubated MCR score of silicon-organic factor1 may reach up to 9×10¹³, up to 7×10¹³, or up to 6×10¹³.

An absolute non-incubated MCR score of silicon-inorganic factor1 may be up to 1×10¹³, up to 5×10¹², or up to 3×10¹². Optionally, this score may be at least 0.5×10¹².

A relative non-incubated MCR score ratio of silicon-inorganic factor1 may be up to 5, up to 3, or up to 1.5. Optionally, this score ratio is at least 0.1, or at least 0.2.

In some embodiments of the pharmaceutical container, the ToF-SIMS data include n datasets consisting of ion-specific masses and their corresponding intensities such that a ToF-SIMS result measured in a specific coating or container can be attributed a specific position in an n-dimensional compositional space, wherein one or more factors, selected from lipid factor1, silicon-organic factor1, silicon-inorganic factor1 and organic factor1, have a factor-specific MCR loading, indicating a conceptional component in said n-dimensional compositional space which can be attributed to said one or more factors, wherein the factor-specific MCR loading characterizes the one or more factors by listing the ions that contribute to the definition of said factor, wherein each absolute LNP-incubated MCR score or relative LNP-incubated MCR score ratio represents the abundance of the corresponding MCR factor in the coating or container. Provided that a (pre-)selected ion species has zero intensity or has a value of zero in the loading of a factor, then that ion species will not contribute to the loading and the related factor.

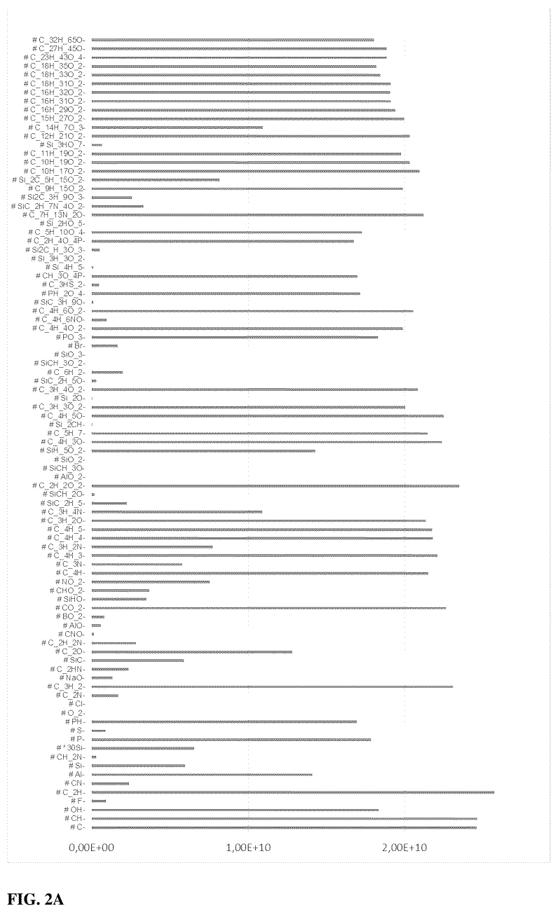

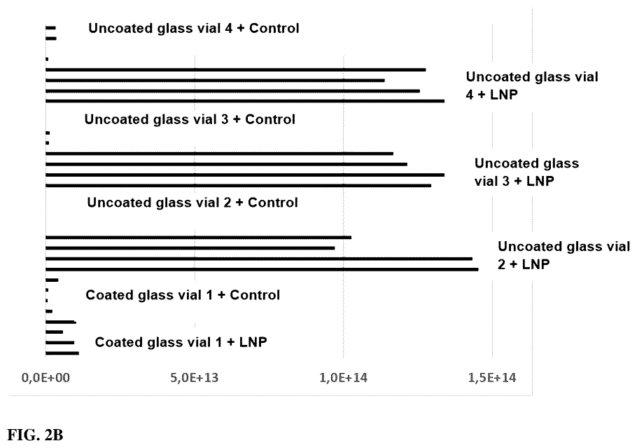

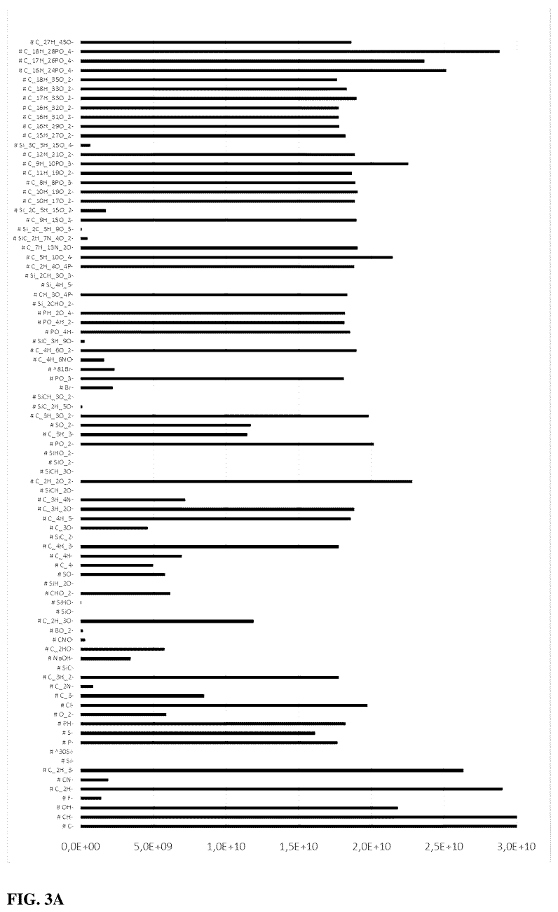

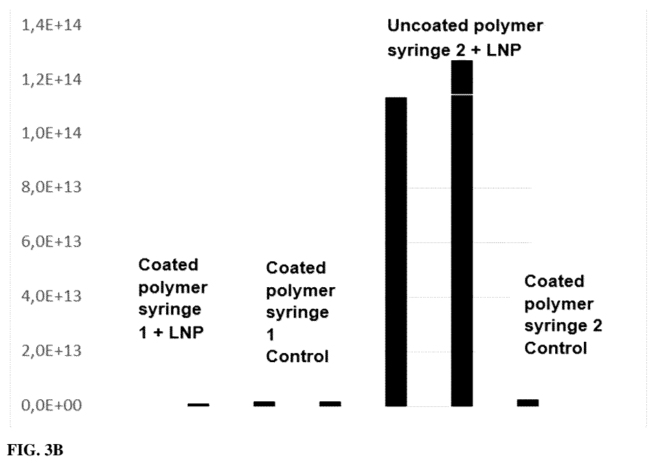

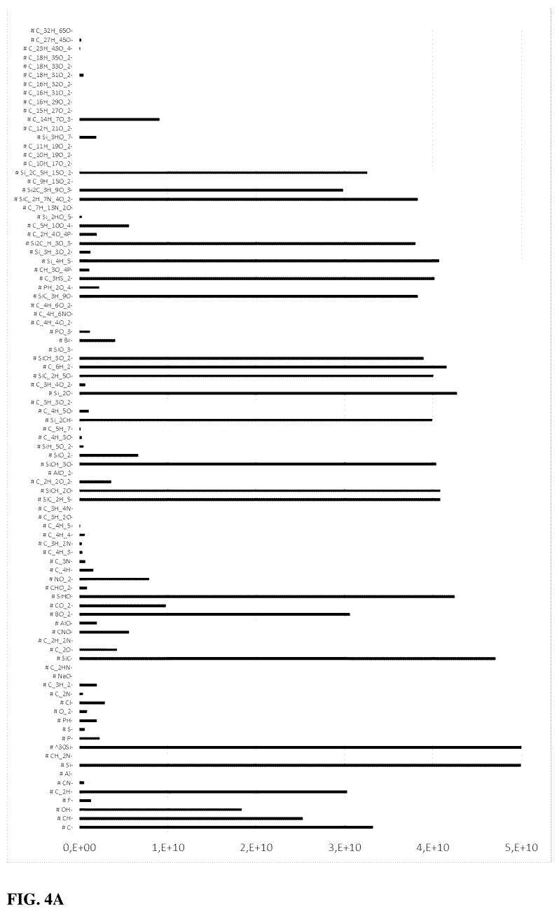

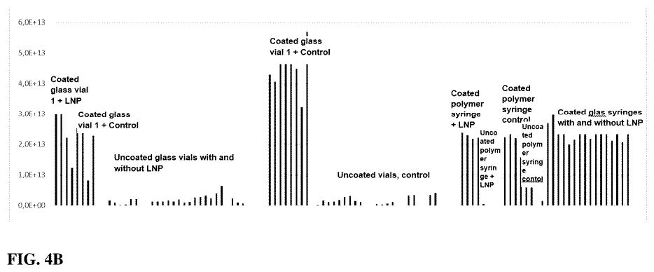

FIG. 2A, FIG. 3A and FIG. 4A show MCR data generated from a bundle of ToF-SIMS spectra representing the n datasets consisting of ion-specific masses. FIG. 2A and FIG. 3A show the loading and score for the characteristic lipid factor1 obtained from the data matrix of negative-ToF-SIMS spectra for adsorbed lipid-containing compounds. FIG. 4A shows the loading for the characteristic silicon-organic factor1 obtained from the data matrix of negative-ToF-SIMS spectra and FIG. 4B the score for adsorbed silicon-organic compounds on the inner surface of a container.

The one or more factors may be selected from lipid factor1, silicon-organic factor1, silicon-inorganic factor1 and organic factor1, wherein each of the factors has a factor-specific MCR loading which indicates a conceptional component in said n-dimensional compositional space which can be attributed to said one or more factors, wherein the factor-specific MCR loading characterizes each factor by listing the ions that contribute to the definition of said factor. The conceptional component corresponds to compound classes, such as e.g. lipids, siloxanes, and glass-typical species like silicon. As such the conceptional component is not present in the coating or on the container.

Lipid factor1 generally correlates with the presence of lipids, e.g. when the MCR score of lipid factor1 is high, the abundance of lipids is interpreted as high including in the assessment of additional information from the MCR scores of the other aforementioned factors. In some embodiments, the lipid factor1 includes, in its factor specific MCR loading, one or more of the following ions:
fatty acid-ions;
[CₙH₂ₙ₋₁O₂]⁻, wherein n is 10, 12, 14, 16 or 18;
[CₙH₂ₙ₋₃O₂]⁻, wherein n is 10, 12, 14, 16 or 18;
[CₙH₂ₙ₋₅O₂]⁻, wherein n is 16 or 18;
phosphatidyl-choline ions;
[(CH)ₙH₂O_{4P}]⁻, wherein n=0, 1, 2 or 3.

Silicon-organic factor1 generally correlates with the presence of silicon-organic compounds, e.g. when the MCR score of silicon-organic factor1 is high, the abundance of silicon-organic compounds is interpreted as high, including in the assessment of additional information from the MCR scores of the other aforementioned factors. In some embodiments, the silicon-organic factor1 includes, in its factor specific MCR loading, one or more of the following ions:
silane species,
silicon-carbon species (i.e. species having Si and C atoms),
polysiloxane species having the formula [OSiR₁R₂]ₙ⁻, wherein R₁ and R₂ are independently selected from methyl, ethyl, propyl, and wherein n is any integer between 2 and 10.

Silicon-inorganic factor1 generally correlates with the presence of inorganic compounds, e.g. when the MCR score of silicon-inorganic factor1 is interpreted as high including in the assessment additional information from the MCR scores of the other aforementioned factors, the abundance of inorganic compounds (e.g. glass components, or inorganic oxides) is high. In some embodiments, the silicon-inorganic factor1 includes, in its factor specific MCR loading, one or more of the following ions:
silicon species and/or silicon oxide species;
aluminium oxide species and/or boron species and/or boron oxide species;
halogen species.
alkali oxide species and/or earth alkali oxide species.

In some embodiments, the lipid factor1 includes, in its factor specific MCR loading, one or more of the following ions: [C₁₀H₁₇O₂]⁻, [C₁₀H₁₉O₂]⁻, [C₁₂H₂₁O₂]⁻, [C₁₆H₂₉O₂]⁻, [C₁₆H₃₁O₂]⁻, [C₁₆H₃₂O₂]⁻, [C₁₈H₃₁O₂]⁻, [C₁₈H₃₃O₂]⁻, [C₁₈H₃₅O₂], [PO₃]⁻, [PH₂O₄]⁻, [CH₃O₄P]⁻, [C₂H₄O₄P]⁻.

In some embodiments, the silicon-organic factor1 includes, in its factor specific MCR loading, one or more of the following ions: [SiC]⁻, [SiCH₃O]⁻, [SiCH₃O₂]⁻, [SiC₂H₅O]⁻, [Si₂CHO₂]⁻, [SiC₃H₉O]⁻, [Si₂C₅H₁₅O₂]⁻, [Si₃C₅H₁₅O₄]⁻.

In some embodiments, the silicon-inorganic factor1 includes, in its factor specific MCR loading, one or more of the following ions: OH⁻, Al⁻, Si⁻, P⁻, Cl⁻, NaO⁻, AlO⁻, BO₂⁻, SiHO⁻, AlO₂⁻, SiO₂⁻, SiH₅O₂⁻, Si₃H₃O₂⁻, Si₂HO₅⁻.

In some embodiments, the lipid factor1 includes, in its factor specific MCR loading, at least five of the following ions: [C₁₀H₁₇O₂]⁻, [C₁₀H₁₉O₂]⁻, [C₁₂H₂₁O₂]⁻, [C₁₆H₂₉O₂]⁻, [C₁₆H₃₁O₂]⁻, [C₁₆H₃₂O₂]⁻, [C₁₈H₃₁O₂]⁻, [C₁₈H₃₃O₂]⁻, [C₁₈H₃₅O₂]⁻, [PO₃]⁻, [PH₂O₄]⁻, [CH₃O₄P]⁻, [C₂H₄O₄P]⁻.

In some embodiments, the silicon-organic factor1 includes, in its factor specific MCR loading, at least four of the following ions: [SiC]⁻, [SiCH₃O]⁻, [SiCH₃O₂]⁻, [SiC₂H₅O]⁻, [Si₂CHO₂]⁻, [SiC₃H₉O]⁻, [Si₂C₅H₁₅O₂]⁻, [Si₃C₅H₁₅O₄]⁻.

In some embodiments, the silicon-inorganic factor1 includes, in its factor specific MCR loading, at least five of the following ions: OH⁻, Al⁻, Si⁻, P⁻, Cl⁻, NaO⁻, AlO⁻, BO₂⁻, SiHO⁻, AlO₂⁻, SiO₂⁻, SiH₅O₂⁻, Si₃H₃O₂⁻, Si₂HO₅⁻.

In some embodiments, the lipid factor1 includes, in its factor specific MCR loading, the following ions: [C₁₀H₁₉O₂]⁻, [C₁₂H₂₁O₂]⁻, [C₁₆H₂₉O₂]⁻, [C₁₆H₃₁O₂]⁻, and [C₁₈H₃₅O₂]⁻.

In some embodiments, the silicon-organic factor1 includes, in its factor specific MCR loading, the following ions: [SiCH₃O]⁻, [SiCH₃O₂]⁻, [SiC₂H₅O]⁻, [SiC₃H₉O]⁻, and [Si₂C₅H₁₅O₂]⁻.

In some embodiments, the silicon-inorganic factor1 includes, in its factor specific MCR loading, the following ions: OH⁻, Si⁻, SiO₂⁻, SiH₅O₂⁻, and Si₃H₃O₂⁻.

In some embodiments, the MCR scores are calculated using an MCR with a total of 3, 4 or 5 MCR-factors.

In some embodiments, the pharmaceutical container comprises an inner surface and an outer surface, wherein at least part of the inner surface is coated with a coating, wherein on the coated inner surface the container fulfills one or more of the following conditions:
an LNP incubated haze value of less than 50%, or less than 30%, measured according to the ASTM D 1003-13 standard using illuminant D65 and 2° observer, wherein the LNP-incubated haze value is obtained after freezing to −80° C., and incubating for 4 weeks at −80° C.; and/or
a water contact angle of at least 105°, measured according to DIN 55660-2-2011-12.

In some embodiments, the haze value is less than 50%, less than 40%, less than 30%, or less than 20%, measured according to the ASTM D 1003-13 standard using illuminant D65 and 2° observer. In some embodiments, the haze value is at least 1%, at least 2%, at least 3%, or at least 5%, measured according to the ASTM D 1003-13 standard using illuminant D65 and 2° observer. The LNP incubated haze value is determined after incubating the containers with an LNP composition, wherein the LNP-incubated haze value is obtained after freezing to −80° C., and incubating for 4 weeks at −80° C. Other than that, the treatment is as described above of the LNP incubated MCR scores.

In some embodiments, the water contact angle is at least 105°, or at least 110° measured according to DIN 55660-2-2011-12. In some embodiments, the water contact angle is 125° or less, or 120° or less, measured according to DIN 55660-2-2011-12. The water contact angle is determined on the container without prior LNP incubation.

In some embodiments, the container is a glass container or a polymer container.

In some embodiments, the container comprises a cyclic olefin copolymer. In some embodiments, the container comprises a cyclic olefin polymer.

In some embodiments, the container has one or more of the following properties:
a wall thickness between 0.50 and 10.0 mm, or between 1.00 and 4.00 mm; and/or
a volume capacity of the container of 0.1 ml to 1000 ml, 0.5 ml to 500 ml, 1 ml to 250 ml, 2 ml to 30 ml, 2 ml to 15 ml, or about 1 ml, 2 ml, 3 ml, 4, ml, 5 ml, 6 ml, 7 ml, 8 ml, 9 ml, 10 ml, 11 ml, 12 ml, 13 ml, 14 ml or 15 ml; optionally from 5 to 15 ml.

In some embodiments, the container has a wall thickness of 0.50 mm or more, 1.00 mm or more, or 2.0 mm or more. In some embodiments, the container has a wall thickness of 10.0 mm or less, or 7.00 mm or less, or 4.0 mm or less.

In some embodiments, the container is a syringe, a cartridge, an ampoule or a vial.

Glass Composition

In some embodiments, the container comprises a glass composition comprising 50 to 90 wt. % SiO₂, and 3 to 25 wt. % B₂O₃.

In some embodiments, the container comprises a glass composition comprising aluminosilicate, optionally comprising 55 to 75 wt. % SiO₂, and 11.0 to 25.0 wt. % Al₂O₃.

In some embodiments, the container comprises a glass composition comprising 70 to 81 wt. % SiO₂, 1 to 10 wt. % Al₂O₃, 6 to 14 wt. B₂O₃, 3 to 10 wt. % Na₂O, 0 to 3 wt. % K₂O, 0 to 1 wt. % Li₂O, 0 to 3 wt. % MgO, 0 to 3 wt. % CaO, and 0 to 5 wt. % BaO.

In some embodiments, the container comprises a glass composition comprising 72 to 82 wt. % SiO₂, 5 to 8 wt. % Al₂O₃, 3 to 6 wt. B₂O₃, 2 to 6 wt. % Na₂O, 3 to 9 wt. % K₂O, 0 to 1 wt. % Li₂O, 0 to 1 wt. % MgO, and 0 to 1 wt. % CaO.

In some embodiments, the container comprises a glass composition comprising 60 to 78 wt. % SiO₂, 7 to 15 wt. B₂O₃, 0 to 4 wt. % Na₂O, 3 to 12 wt. % K₂O, 0 to 2 wt. % Li₂O, 0 to 2 wt. % MgO, 0 to 2 wt. % CaO, 0 to 3 wt. % BaO, and 4 to 9 wt. % ZrO₂.

In some embodiments, the container comprises a glass composition comprising 50 to 70 Wt.% SiO₂, 10 to 26 wt. % Al₂O₃, 1 to 14 wt. B₂O₃, 0 to 15 wt. % MgO, 2 to 12 wt. % CaO, 0 to 10 wt. % BaO, 0 to 2 wt. % SrO, 0 to 8 wt. % ZnO, and 0 to 2 wt. % ZrO₂.

In some embodiments, the container comprises a glass composition comprising 55 to 70 wt. % SiO₂, 11 to 25 wt. % Al₂O₃, 0 to 10 wt. % MgO, 1 to 20 wt. % CaO, 0 to 10 wt. % BaO, 0 to 8.5 wt. % SrO, 0 to 5 wt. % ZnO, 0 to 5 wt. % ZrO₂, and 0 to 5 wt. % TiO₂.

In some embodiments, the container comprises a glass composition comprising 65 to 72 wt. % SiO₂, 11 to 17 wt. % Al₂O₃, 0.1 to 8 wt. % Na₂O, 0 to 8 wt. % K₂O. 3 to 8 wt. % MgO, 4 to 12 wt. % CaO, and 0 to 10 wt. % ZnO.

In some embodiments, the container comprises a glass composition comprising 64 to 78 wt. % SiO₂, 4 to 14 wt. % Al₂O₃, 0 to 4 wt. % B₂O₃, 6 to 14 wt. % Na₂O, 0 to 3 wt. % K₂O, 0 to 10 wt. % MgO, 0 to 15 wt. % CaO, 0 to 2 wt. % ZrO₂, and 0 to 2 wt. % TiO₂.

Coating

In some embodiments, the coating comprises the elemental species Si, C, O and H.

In some embodiments, the coating comprises at least one layer having a carbon content of at least 55%.

In some embodiments, the coating is derived and/or generated from one or more of hexamethyldisiloxane (HMIDSO), hexamethyldisilazane (HMDS), tetramethylsilane (TMS), trimethylborazole (TMB), tri(dimethylaminosilyl)-amino-di(dimethylamino)borane (TDADB), tris(trimethylsilyl)borate (TMSB), hexamethylcyclotrisiloxan (HMCTSO), octamethylcyclotetrasiloxan (OMCTS), decamethylcyclopentasiloxan (DMCPS), dodecamethylcyclohexasiloxan (DMCHS), diacetoxy-di-t-butoxysilane (DADBS), tetraethoxysilane (TEOS), tris(trimethylsilyloxy)vinylsilane (TTMSVS), vinyltriethoxysilane (VTES) and/or combinations thereof.

In some embodiments, the coating comprises at least one layer, wherein the coating, or at least one layer of the coating, fulfills the following parameter:
[Si₂C₅H₁₅O₂⁻]₂₀/[Si₂C₅H₁₅O₂⁻]₈₀≥x1_{[Si2C5H15O2-]};
wherein [Si₂C₅H₁₅O₂⁻]₂₀ are the counts of [Si₂C₅H₁₅O₂⁻] ions, measured by a TOF-SIMS, at 20% of the time a sputter gun beam needs to reach the glass surface; and
wherein [Si₂C₅H₁₅O₂⁻]₈₀ are the counts of [Si₂C₅H₁₅O₂⁻]₈₀ ions, measured by a TOF-SIMS, at 80% of the time a sputter gun beam needs to reach the glass surface,
wherein x1_{[Si2C5H15O2-]} is 1.2, 1.5, 2, 3, 5, 8, or 12.

In some embodiments, the coating comprises at least one layer, wherein the at least one layer of the coating fulfills the following parameter(s):
[Si₂C₃H₉O₃⁻]₂₀/[Si₂C₃H₉O₃⁻]₈₀≤x1_{[Si2C3H9O3-]-},
wherein x1_{[Si2C3H9O3-]} is 1.1, optionally 1.5, optionally 2, optionally 3; and/or
[Si₂C₃H₉O₃⁻]₂₀/[Si₂C₃H₉O₃⁻]₈₀≤x2_{[Si2C3H9O3-]-},
wherein x2_{[Si2C3H9O3-]} is 100, optionally 75, optionally 50, optionally 40, optionally 30, optionally 20, optionally 10, optionally 8, optionally 6, optionally 5, optionally 4,
wherein [Si₂C₃H₉O₃⁻]₂₀ are the counts of [Si₂C₃H₉O₃⁻] ions, measured by a TOF-SIMS, at 20% of the time a sputter gun beam needs to reach the glass surface, and
wherein [Si₂C₃H₉O₃⁻]₈₀ are the counts of [Si₂C₃H₉O₃⁻]₈₀ ions, measured by a TOF-SIMS, at 80% of the time a sputter gun beam needs to reach the glass surface.

For this analysis a ToF-SIMS depth profiling measuring process is used, wherein the start was set to 0% of the time the sputter analysis process needed to reach the glass surface. At this point the ratio of the counts of [Al⁺] ions to the counts of [Si⁺] ions may be optionally 0.00. After a certain analysis time (sputter time), the value of the ratio of the counts of [Al⁺] ions to the counts of [Si⁺] ions is 0.10 or more. This point indicates the time a sputter gun beam needs to reach the glass surface as aluminum is normally assigned as a glass element. This point is set to 100% relating to 100% of the time the sputter analysis process needed to reach the glass surface.

The ToF-SIMS depth profiling process used for this measurement is different from the static ToF-SIMS method used to obtain the datasets for the MCR.

Filled Pharmaceutical Container

In some embodiments, the invention provides a filled pharmaceutical container comprising the pharmaceutical container provided according to this disclosure, and a pharmaceutical composition comprising lipid-based carrier systems, in particular lipid nanoparticles.

In some embodiments, the lipid-based carrier systems or the lipid nanoparticles comprise one or more of the following compound classes:
a) a phospholipid; and/or
b) cholesterol or a steroid functionalised at the C²⁴ position with a linear or branched alkyl chain, wherein the linear or branched alkyl chain comprises 1 to 50 C atoms, wherein the C²⁴ position is defined according to IUPAC nomenclature and/or
c) a PEG-modified lipid; and/or
d) a cationic lipid.

In some embodiments, the pharmaceutical composition is liquid or a frozen liquid and comprises
a) 1.05 mg/mL to 1.95 mg/mL phospholipid; and/or
b) 2.3 mg/mL to 4.3 mg/mL cholesterol; and/or
c) 0.56 mg/mL to 1.05 mg/mL PEG-modified lipid; and/or
d) 0.50 mg/mL to 9.40 mg/mL cationic lipid.

In some embodiments, the lipid nanoparticles are characterized by one or more of the following properties:
i) a polydispersity index (PDI) value <0.5, or ≤0.1; and
ii) a z-average diameter of up to 200 nm, up to 150 nm, or up to 100 nm, such as 10 nm to 200 nm, 20 nm to 150 nm, or 50 to 100 nm, measured with Dynamic Light Scattering (DLS) using a Malvern zetasizer.

In some embodiments, the z-average diameter of the lipid nanoparticles is 10 nm or more, 20 nm or more, or 50 nm or more.

The particle size, and PDI were determined at room temperature using a zetasizer Nano ZS from Malvern™ (Malvern Instruments Ltd., Worcestershire, United Kingdom). Size and PDI were measured after dilution to a lipid concentration of 0.07 mg/ml with a 10 mM phosphate buffer with a pH of 7.4 using the automatic mode. The default settings of the automatic mode of the zetasizer Nano ZS from Malvern™ (Malvern Instruments Ltd., Worcestershire, United Kingdom) were the following: number of measurements=3; run duration=60 s; number of runs=10; equilibration time=60 s; refractive index solvent 1.45; refractive index dispersant 1.335; viscosity=1.02 cP; temperature=24.9° C.; dielectric constant=78.5 F/m; backscattering mode (173°); automatic voltage selection; Smoluchowski equation.

In some embodiments, the pharmaceutical composition comprises RNA, e.g. mRNA or siRNA or saRNA.

Use

In some embodiments, the invention relates to the use of the pharmaceutical container for the storage and/or transport of pharmaceutical compositions comprising lipid-based carrier systems, in particular lipid nanoparticles.

The (filled) pharmaceutical container may advantageously display less adhesion of ingredients of pharmaceutical composition to the inner surface of the container. Advantageously, when the pharmaceutical composition comprises lipid-based carrier systems, in particular lipid nanoparticles, there may be less adhesion of the lipids and/or lipid nanoparticles to the inner surface of the container. Thus, dose uniformity and an unaltered pharmaceutical composition are safeguarded.

Lipid Nanoparticles (LNPs)

In this disclosure “lipid-based carrier system” includes lipid-containing drug delivery systems, such as liposomes, micelles, SEDDS and lipid nanoparticles.

Solid lipid nanoparticles or lipid nanoparticles (LNPs) are nanoparticles composed of lipids.

In some embodiments, the lipid nanoparticles comprise one or more of the ionisable lipids disclosed in Table 2 of Buschmann et al. (Vaccines 9, 2021, 65), which is herein incorporated by reference.

In some embodiments, the lipid nanoparticles comprise a PEG-lipid, wherein the PEG-lipid is obtainable by PEGylation of a lipid.

In the context of the present disclosure, PEGylation refers to the process of covalent and non-covalent attachment of polyethylene glycol (PEG) polymer chains to (macro)molecules, such as e.g. lipids, which are then PEGylated.

In some embodiments, the lipid nanoparticles comprise one or more of the cationic lipids disclosed in Table 2 and Table 3 of WO 2017/075531 A1, which is herein incorporated by reference.

In the context of the present disclosure, a lipid may be understood as one of the four compounds or compound classes: cholesterol; fatty acids of a chain length of 12 carbon atoms or more up to 26 carbon atoms; triglycerides based on the condensation product of glycerin and three fatty acids which may be the same or different; sphingolipids and phospholids.

In some embodiments, the lipid nanoparticles comprise an ionisable lipid, DSPC (di-stearoylphosphatidylcholine), cholesterol, and a PEG-lipid.

In some embodiments, the lipid nanoparticles additionally comprise polynucleotides, in particular RNA.

Methods

Glass Container Preparation

A coated glass container and an uncoated glass container (the latter serving as a reference container), both having the same dimensions, same glass type and glass composition, are treated under the exact same conditions.

For measuring the LNP-incubated MCR scores, the respective container is cleaned with UltraPure water (purity 1 analogue DIN ISO 3696 with ≤0.1 μS/cm at 25° C.) and dried under laminar-flow conditions. The container is then filled with a reference LNP-composition, frozen to −80° C., incubated for 12 hours at −80° C., and then thawed to 5° C. within 12 hours.

In a first variant, the reference LNP-composition is the Comirnaty vaccine (license number EU/1/20/1528).

In a second variant, the reference-LNP contains the following lipids in the indicated amounts: 7.2 mg/mL (4-hydroxybutyl)azanediyl)bis(hexane-6,1-diyl)bis(2-hexyldecanoate), 0.83 mg/mL 2[(polyethylene glycol)-2000]-N,N-ditetradecylacetamide, 1.5 mg/mL 1,2-distearoyl-sn-glycero-3-phosphocholine, and 3.3 mg/mL cholesterol, in phosphate-buffered saline (PBS) (pH 7.4) with a saccharide content of 10 wt. % and the following concentrations.

| Compound | Concentration | Concentration |
|---|---|---|
| (salt or sugar) | (mmol/L) | (g/L) |
| NaCl | 137 | 8.0 |
| KCl | 2.7 | 0.2 |
| Na₂HPO₄ | 10 | 1.42 |
| KH₂PO₄ | 1.8 | 0.24 |
| Sucrose | | 100 |

Both containers are analyzed via ToF-SIMS and MCR analysis (see next sections).

An absolute LNP-incubated MCR score means the MCR score obtained from a single container, either the coated glass container of the uncoated (reference) glass container, for one specific factor, wherein the factor is selected from lipid factor1, silicon-organic factor1, silicon-inorganic factor1 and organic factor1, each factor having a factor-specific MCR loading.

The relative LNP-incubated MCR score ratio refers to the quotient of the MCR score of a specific factor between the coated glass container and the uncoated (reference) glass container.

Polymer Container Preparation

A coated polymer container and an uncoated polymer container, both having the same dimensions, same glass type and glass composition, are treated under the exact same conditions.

The polymer container preparation is the same as for the glass container preparation, except that the cleaning with UltraPure water (purity 1 analogue DIN ISO 3696 with ≤0.1 μS/cm at 25° C.) and drying under laminar-flow conditions is omitted.

ToF-SIMS (Time-of-Flight Secondary Ion Mass Spectrometry)

In the following the measuring method and the data evaluation of the specific ToF-SIMS measurement is explained in detail. For the measurement a TOF.SIMS 4 from Iontof was used. If not stated otherwise, the ToF-SIMS are measured according to ASTM E 1829 und ASTM E 2695.

Measurement

The following parameter settings were used for the ToF-SIMS analysis:
primary ion: Ga⁺; (alternatively using a TOF.SIMS 5 and Bi⁺);
(primary ion) energy: 25000V;
measuring area: 100×100 μm²;
primary ion dose density: 6×10¹² cm²;
surface discharge: via low-energetic electrons.

Spectral data were acquired for 100 s with subsequent integration.

Sample Preparation

Each sample of a (coated) container was cut lengthwise in two pieces and positioned in such a way that the centerline of the primary ion gun of the ToF-SIMS apparatus hit the inner surface of the sample. The thus generated intrinsically ionised secondary ions have been analysed via Time-of-flight analysis and separated into different detectable mass/charge ratios. Accordingly, a high-resolution mass spectrum (Δm/m>3000 for Si) is obtained covering both atomic and molecular ion species. Die surface sensitivity covers several few monolayers.

Data Preparation

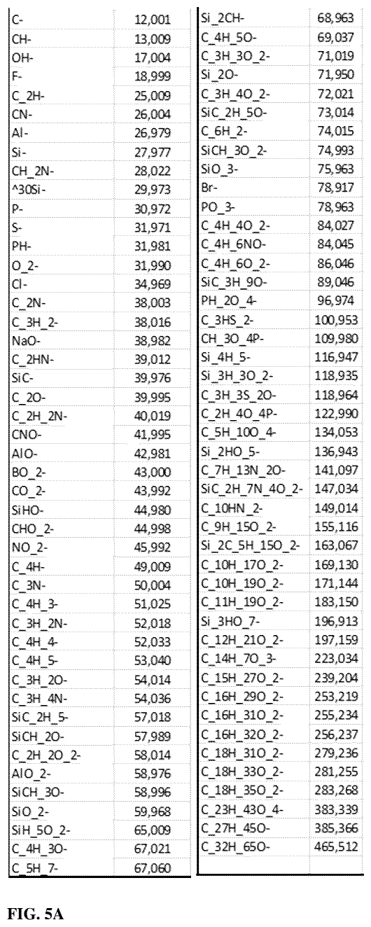

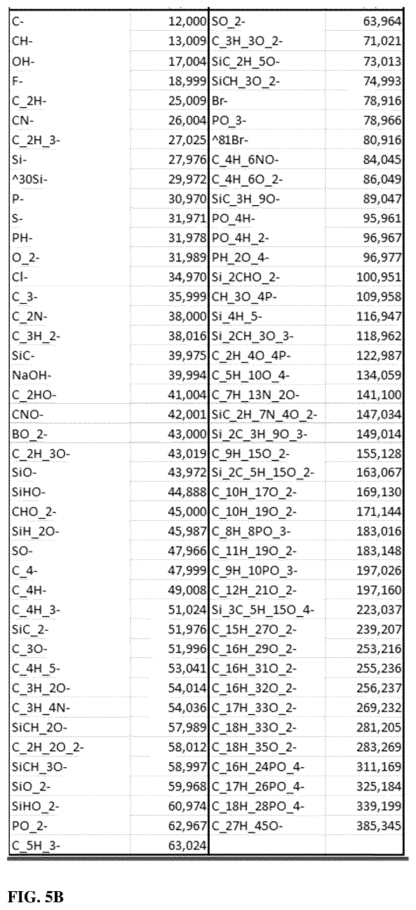

The ToF-SIMS data include n datasets consisting of ion-specific masses and their corresponding intensities. Ion species/masses are selected from the raw ToF-SIMS data, including their intensities, as indicated in FIG. 5A and FIG. 5B for, respectively, a glass container and a polymer container. The ion-specific masses relate to negative ions only because the experiment is done in negative mode. For interpretation of the masses, the spectra library from IONTOF GmbH can be used. Every ion species/mass is normalised according to its individual variance.

Multivariate Curve Resolution (MCR)

For the identification of MCR factors, the ToF-SIMS result are subjected to a subsequent multivariate analysis via MCR (Multivariate Curve Resolution). MCR is a statistical analysis method, which in its most general approach decomposes a two-way data matrix D (m×n) into two matrices C (m×k) and S^{T} (k×n), containing respectively pure concentration profiles and pure spectra of the k species of an unknown mixture, according to the equation D=CS^{T}+E, wherein E is an error matrix containing the residuals of the data (Ruckebusch & Blanchet, Analytica Chimica Acta 765, 2013, 28-36). The MCR method has been adapted to decompose and analyse ToF-SIMS. Commercially available software can be used for this task, e.g. the software package SurfaceLab Ver 7.1., wherein optionally the number of factors is set to 3, 4 or 5. A general account of how spectral information can be dissected is given by Juan & Tauler (Analytica Chimica Acta 1145, 2021, 59-78).

Summing up, the ToF-SIMS result measured in a specific coating or container can be attributed a specific position in an n-dimensional compositional space. MCR is used to reduce the complexity of the ToF-SIMS result by summarizing the datasets into a more limited number of variables, the so-called “factors”. The results of the MCR are a set of factors, loadings and corresponding scores. Each of the factors has a factor-specific MCR loading, indicating a conceptional component in said n-dimensional compositional space which can be attributed to said factor, wherein the loading characterizes the factor in that it lists the ions that contribute to the definition of said factor. Each factor relates to substances present in or on the coating or container. To be clear, the conceptional component is not in fact present in the coating or container. Each score indicates the intensity of the corresponding factor. It correlates with the abundance of substances in or on the coating or container.

ToF-SIMS Depth Profiling Using a Sputter Gun

Depth profiling values for the ions, e.g. the ions [Al⁺]₂₀, [Al⁺]₈₀, [Si₂C₅H₁₅O₂⁻]₂₀ and [Si₂C₅H₁₅O₂⁻]₈₀, can be obtained according to the following method.

Measuring Method

For the measurement a TOF SIMS (TOF.SIMS 5 from Iontof) can be used. If not stated otherwise, the TOF-SIMS are measured according to ASTM E 1829 und ASTM E 2695.

The following parameter settings were used for the TOF-SIMS:

### For the Analysis:

primary ion: Bi³⁺;
energy: 30000 eV;
measuring area: 200×200 μm²;
pattern: 128×128 random; and
bismuth analysis current: 0.3 pA.

### For the Sputter Gun (Argon Cluster Source):

sputter ion: Ar1051 (Argon);
energy: 5000 eV;
sputter area: 500×500 μm²; and
sputter current of Ar-cluster source: 1 nA.

### Furthermore:

cycle time: 200 μs
analyzer extractor: 2160V;
analyzer detector: 9000V;
charge compensation: flood gun;
primary ion flight time correction: on; and
gas Flooding: 9×10⁻⁷ mbar.

A sample of a coated glass element, for example one half of an on the inside coated container, which was cut lengthwise in two pieces, is positioned in such a way that the centerline of the sputter gun and the centerline of the liquid metal ion gun of TOF-SIMS hit the coated area of the sample so that the sputter area covers the entire measuring area, optionally that the centerline of the sputter gun and the centerline of the liquid metal ion gun of TOF-SIMS hit the same point of the coated area of the sample. The TOF-SIMS measures either positive ions or negative ions. To obtain both kinds of ions, two measurements can be conducted using for each measurement a new area of the same sample or a new sample, e.g. the first and the second half of a coated container, which is cut lengthwise in two pieces.

Data Evaluation

For the data evaluation all counts of ions are normalized to the [Si⁺] ions and [Si⁻] ions, respectively, whereby the [Si⁺] ions and [Si⁻] ions, respectively, are set to 1.

Additionally the point (sputter time), when the TOF SIMS analysis starts, is set to 0% and the point, when the glass surface is reached, is set to 100%. This is indicated by the [Al⁺] signal and the [AlO₂⁻] signal, respectively, as those signals can be clearly assigned to the glass.

In the case positive ions are measured, the point, when the sputter gun reaches the glass surface, may be, optionally is, the point, when the ratio of the counts of [Al⁺] ions to the counts of [Si⁺] ions is equal to or exceeds a value of 0.10 for the first time.

In the case negative ions are measured, the point, when the sputter gun reaches the glass surface, may be, optionally is, the point, when the ratio of the counts of [AlO₂⁺] ions to the counts of [Si⁻] ions is equal to or exceeds a value of 0.10 for the first time.

The point when the analysis, i.e. the measuring process, was started, was set to 0% of the time the sputter analysis process needed to reach the glass surface. At this point the ratio of the counts of [Al⁺] ions to the counts of [Si⁺] ions may be, optionally is, 0.00. After a certain analysis time (sputter time), the value of the ratio of the counts of [Al⁺] ions to the counts of [Si⁺] ions is 0.10 or more. This point indicates the time a sputter gun beam needs to reach the glass surface as aluminum is clearly assigned as glass element. Until this point, the ratio was never 0.10 or higher. Consequently, this point was set to 100%, since this is 100% of the time the sputter analysis process needed to reach the glass surface.

### Examples

Coated Glass Vial

A 20 R vial (glass vial manufactured with glass tubing, Fiolax® clear, Schott AG, Germany) was provided. As first pretreatment, a washing pretreatment was performed in which the vial was washed with ultrapure water with ≤10 μS/cm at 25° C. for two minutes at room temperature, for 6 minutes at 40° C., and subsequently for 25 minutes at room temperature in a laboratory dishwasher (LS-2000 from HAMO AG). Afterwards, the vial was dried for 20 minutes at 300° C.

Subsequently, the vial was treated and coated simultaneously using an apparatus according to WO 03/015122 A1. For all plasma treatments, a microwave irradiation was used having a frequency of 2.45 GHz. The reaction chamber was the inside of the vial. Ambient conditions prevailed outside of the vial.

First, the inside of the vial was evacuated until a value of 0.05 mbar was reached. Afterwards, oxygen was filled in the vial, at a flow rate of 25 sccm, until a pressure of 5 mbar was reached and then a plasma pretreatment was started. The plasma was excited with an input power of 5500 W in a pulsed mode with a pulse duration of 0.5 ms, and pulse pause of 1.8 ms. The plasma pretreatment was performed for 17 seconds until the temperature of the vial was 250° C., and measured with a pyrometer at the middle of the cylindrical part of the vial.

Immediately afterwards the coating process was performed. The vial was filled with HMDSO (hexamethyldisiloxane), at a flow rate of 12.5 sccm, and the pressure was set to 0.8 mbar. Then, the vial was irradiated for 0.2 s (pressure: 0.8 mbar, flow rate 12.5 sccm HMDSO, input power: 6000 W, pulse duration: 0.050 ms, pulse pause: 30 ms) and subsequently irradiated for 13 s (pressure: 0.8 mbar, flow: 12.5 sccm HMDSO, input power: 4500 W, pulse duration: 0.008 ms, pulse pause: 1 ms).

Afterwards, a post-processing was performed, i.e. filling the vial with oxygen and cooling the vial to room temperature in the presence of oxygen to obtain a coated vial.

Alternatively, the coating may be prepared as described in EP 21164784.7, which is herein incorporated by reference.

Coated Polymer Vials

Polymer syringes made of COC (cyclic olefin copolymer) of the Luer Lock format (1 mL volume) were used. A silicon-containing fluid was applied on the inner surface of the container, via a spray process or a bath process. The silicon-containing fluid may be a mixture of different silicon-organic compounds, such as poly(organo)siloxanes, wherein at least one reactive component may be thermally cured to form a network.

Alternatively, the coating may be prepared analogously and according to the description in EP 21164784.7, which is herein incorporated by reference.

Reference Lipid Nanoparticles (LNP)

The coated glass vial was treated with either a phosphate-buffered saline (PBS) solution or a Reference-LNP in the same PBS solution. The differently treated coated glass vial were subjected to the above described ToF-SIMS measurement and data were extracted according to the above described MCR analysis.

In a first variant, the reference LNP-composition was the Comirnaty vaccine (license number EU/1/20/1528).

In a second variant, the reference-LNP contained the following lipids in the indicated amounts: 7.2 mg/mL (4-hydroxybutyl)azanediyl)bis(hexane-6,1-diyl)bis(2-hexyldecanoate), 0.83 mg/mL 2[(polyethylene glycol)-2000]-N,N-ditetradecylacetamide, 1.5 mg/mL 1,2-distearoyl-sn-glycero-3-phosphocholine, and 3.3 mg/mL cholesterol, in phosphate-buffered saline (PBS) (pH 7.4) with a saccharide content of 10 wt. % and the following concentrations.

| Compound | Concentration | Concentration |
|---|---|---|
| (salt or sugar) | (mmol/L) | (g/L) |
| NaCl | 137 | 8.0 |
| KCl | 2.7 | 0.2 |
| Na₂HPO₄ | 10 | 1.42 |
| KH₂PO₄ | 1.8 | 0.24 |
| Sucrose | | 100 |

Further LNP Formulations

LNPs with similar formulations may alternatively be used which formulations may deviate up to 30 wt. % from the given quantities of individual lipid components.

Additionally the LNPs contain RNA, such as mRNA, particularly based on polynucleotides containing adenine.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A pharmaceutical container comprising: an inner surface and an outer surface, wherein at least part of the inner surface is coated with a coating to form a coated inner surface, wherein on the coated inner surface the container, based on negative mode Time-of-Flight-Secondary Ion Mass Spectrometry (ToF-SIMS) data, has a relative lipid nanoparticle (LNP)-incubated Multivariate Curve Resolution (MCR) score ratio of lipid factor1 of less than 0.67.

2. The pharmaceutical container of claim 1, wherein the container has an absolute LNP-incubated MCR score of lipid factor1 of less than 7×10¹³.

3. The pharmaceutical container of claim 1, wherein the container has:
an absolute LNP-incubated MCR score of silicon-organic factor1 of at least 1×10¹²; and/or
a relative LNP-incubated MCR score ratio of silicon-organic factor1 of at least 2.

4. The pharmaceutical container of claim 1, wherein the pharmaceutical container has:
an absolute LNP-incubated MCR score of silicon-inorganic factor1 of at least 1×10¹³;
and/or a relative LNP-incubated MCR score ratio of silicon-inorganic factor1 of up to 5.

5. The pharmaceutical container of claim 1, wherein the container has:
an absolute LNP-incubated MCR score of organic factor1 of at least 1×10¹²; and/or
a relative LNP-incubated MCR score ratio of organic factor1 of at least 0.2.

6. The pharmaceutical container of claim 1, wherein the ToF-SIMS data include n datasets consisting of ion-specific masses and their corresponding intensities such that a ToF-SIMS result measured in a specific coating or container can be attributed a specific position in an n-dimensional compositional space;
wherein one or more factors selected from lipid factor1, silicon-organic factor1, silicon-inorganic factor1 and organic factor1, have a factor-specific MCR loading, indicating a conceptional component in said n-dimensional compositional space which can be attributed to said one or more factors, wherein the factor-specific MCR loading characterizes the one or more factors by listing the ions that contribute to the definition of said factor;
wherein each absolute LNP-incubated MCR score or relative LNP-incubated MCR score ratio represents the abundance of the corresponding factor in the coating or container.

7. The pharmaceutical container of claim 1, wherein
lipid factor1 includes, in its factor specific MCR loading, one or more of the following ions: fatty acid-ions; [CₙH₂ₙ₋₁O₂]⁻, wherein n is 10, 12, 14, 16 or 18; [CₙH₂ₙ₋₃O₂]⁻, wherein n is 10, 12, 14, 16 or 18; [CₙH₂ₙ₋₅O₂]⁻, wherein n is 16 or 18; phosphatidyl-choline ions; [(CH)ₙH₂O₄P]⁻, wherein n=0, 1, 2 or 3, and/or
silicon-organic factor1 includes, in its factor specific MCR loading, one or more of the following ions: silane species, silicon-carbon species, polysiloxane species based on the formula [OSiR₁R₂]ₙ⁻, wherein R₁ and R₂ are independently selected from methyl, ethyl, propyl, and wherein n is any integer between 2 and 10; and/or
silicon-inorganic factor1 includes, in its factor specific MCR loading, one or more of the following ions: silicon species; silicon oxide species, aluminium-species, aluminium oxide species and/or boron species and/or boron oxide species; halogen species; alkali oxide species; earth alkali oxide species.

8. A pharmaceutical container, comprising: an inner surface and an outer surface, wherein at least part of the inner surface is coated with a coating to form a coated inner surface, wherein on the coated inner surface the container fulfills one or more of the following conditions:
a lipid nanoparticle (LNP)-incubated haze value of less than 50% measured according to the ASTM D 1003-13 standard using illuminant D65 and 2° observer, wherein the LNP-incubation includes freezing to −80° C., and incubating for 4 weeks at −80° C.; and/or
a water contact angle of at least 105°, measured according to DIN 55660-2-2011-12.

9. The pharmaceutical container of claim 8, wherein the container is a glass container or a polymer container.

10. The pharmaceutical container of claim 8, wherein the container has one or more of the following properties:
a wall thickness between 0.50 and 10.0 mm; or
a volume capacity of the container of 0.1 ml to 1000 ml.

11. The pharmaceutical container of claim 8, wherein the container is a syringe, a cartridge, an ampoule or a glass vial.

12. The pharmaceutical container of claim 8, wherein the container comprises
a glass composition comprising 50 to 90 wt. % SiO₂, and 3 to 25 wt. % B₂O₃; or
a glass composition comprising aluminosilicate comprising 55 to 75 wt. % SiO₂, and 11.0 to 25.0 wt. % Al₂O₃.

13. The pharmaceutical container of claim 8, wherein the container comprises a glass composition comprising 70 to 81 wt. % SiO₂, 1 to 10 wt. % Al₂O₃, 6 to 14 wt. B₂O₃, 3 to 10 wt. % Na₂O, 0 to 3 wt. % K₂O, 0 to 1 wt. % Li₂O, 0 to 3 wt. % MgO, 0 to 3 wt. % CaO, 0 to 5 wt. % BaO.

14. The pharmaceutical container of claim 8, wherein the container comprises a glass composition comprising 72 to 82 wt. % SiO₂, 5 to 8 wt. % Al₂O₃, 3 to 6 wt. B₂O₃, 2 to 6 wt. % Na₂O, 3 to 9 wt. % K₂O, 0 to 1 wt. % Li₂O, 0 to 1 wt. % MgO, 0 to 1 wt. % CaO.

15. The pharmaceutical container of claim 8, wherein the container comprises a glass composition comprising 60 to 78 wt. % SiO₂, 7 to 15 wt. B₂O₃, 0 to 4 wt. % Na₂O, 3 to 12 wt. % K₂O, 0 to 2 wt. % Li₂O, 0 to 2 wt. % MgO, 0 to 2 wt. % CaO, 0 to 3 wt. % BaO, 4 to 9 wt. % ZrO₂.

16. The pharmaceutical container of claim 8, wherein the container comprises a glass composition comprising 50 to 70 wt. % SiO₂, 10 to 26 wt. % Al₂O₃, 1 to 14 wt. B₂O₃, 0 to 15 wt. % MgO, 2 to 12 wt. % CaO, 0 to 10 wt. % BaO, 0 to 2 wt. % SrO, 0 to 8 wt. % ZnO, 0 to 2 wt. % ZrO₂.

17. The pharmaceutical container of claim 8, wherein the container comprises a glass composition comprising 55 to 70 wt. % SiO₂, 11 to 25 wt. % Al₂O₃, 0 to 10 wt. % MgO, 1 to 20 wt. % CaO, 0 to 10 wt. % BaO, 0 to 8.5 wt. % SrO, 0 to 5 wt. % ZnO, 0 to 5 wt. % ZrO₂, 0 to 5 wt. % TiO₂.

18. The pharmaceutical container of claim 8, wherein the coating comprises the elemental species Si, C, O and H.

19. The pharmaceutical container of claim 8, wherein the coating comprises at least one layer having a carbon content of at least 55%.

20. A filled pharmaceutical container, comprising:
a pharmaceutical container comprising an inner surface and an outer surface, wherein at least part of the inner surface is coated with a coating to form a coated inner surface, wherein on the coated inner surface the container, based on negative mode Time-of-Flight-Secondary Ion Mass Spectrometry (ToF-SIMS) data, has a relative lipid nanoparticle (LNP)-incubated Multivariate Curve Resolution (MCR) score ratio of lipid factor1 of less than 0.67;
a pharmaceutical composition comprising lipid-based carrier systems disposed in the pharmaceutical container.
